(19) United States
(12) Patent Application Publication
(10) Pub. No.: US 2026/0030760 A1
(43) Pub. Date: Jan. 29, 2026

(54) PROVIDING A RESULTS DATASET

(71) Applicant: Siemens Healthineers AG, Forchheim (DE)

(72) Inventor: Annette Birkhold, Stuttgart (DE)

(21) Appl. No.: 19/270,925

(22) Filed: Jul. 16, 2025

(30) Foreign Application Priority Data
Jul. 26, 2024 (DE) 10 2024 207 064.5

(51) Int. Cl. G06T 7/00; A61B 6/00; A61B 6/50; G06T 7/246; G06V 10/44; G06V 10/62; G06V 10/75; G06V 10/764; G16H 30/40

(52) U.S. Cl. CPC G06T 7/0016; A61B 6/481; A61B 6/504; G06T 7/248; G06V 10/44; G06V 10/62; G06V 10/751; G06V 10/764; G16H 30/40; G06T 2207/10116; G06T 2207/20224; G06T 2207/30101; G06V 2201/031

(57) ABSTRACT

A computer-implemented method for providing a results dataset includes: acquiring an image dataset of an examination object; identifying portions of a hollow organ in the image dataset based on a mapped contrast medium flow; classifying sub-portions of the identified portions into single-fed and multi-fed sub-portions based on a mapped flow direction of the contrast medium flow and an identification of confluences of the identified portions of the hollow organ mapped in the image dataset, wherein sub-portions of the hollow organ arranged downstream relative to a confluence are classified as multi-fed sub-portions; and providing the results dataset based on the image dataset and the classified sub-portions of the hollow organ, wherein the results dataset has a partial dataset for each classified sub-portion of the hollow organ, and wherein, the partial datasets have a dedicated representation of the contrast medium flow in each classified sub-portion of the hollow organ.

The present patent document claims the benefit of German Patent Application No. 10 2024 207 064.5, filed Jul. 26, 2024, which is hereby incorporated by reference in its entirety.

### TECHNICAL FIELD

The present disclosure relates to a computer-implemented method for providing a results dataset, a provision unit, a medical imaging device, and a computer program product.

### BACKGROUND

Arteriovenous malformations (AVM) are vessel malformations where arteries are directly connected to veins via a complex vascular network, in particular a nidus. Understanding these complex vascular networks completely is important for treatment planning, for example, to remove the entire vascular malformation and to be able to prevent a rupture during the treatment. A three-dimensional (3D) or four-dimensional (4D) digital subtraction angiography (DSA) may be used for planning an AVM treatment, for example, a surgical intervention and/or embolization and/or a radiotherapy treatment. Therein, the 3D-DSA or 4D-DSA is carried out while a catheter is arranged in one of the main blood vessels of a brain of the examination object, for example, in the common carotid artery and/or the vertebral artery, in particular, dependent upon a blood supply to the AVM.

However, AVMs may have a blood supply from more than one main blood vessel, for example, on multiple sides. As a result, a single vessel injection with a contrast medium results in an incomplete opacification of the nidus and the 3D dataset of the AVM is insufficient for treatment planning. As a consequence, a plurality of 3D-DSAs and/or 4D-DSAs may be carried out and assembled following a respective reconstruction. Disadvantageously, this increases an X-ray dose to the patient. Furthermore, additional process acts are needed.

A complete opacification of the nidus may also be achieved, for example, by injecting the contrast medium in an ascending thoracic aorta. Alternatively, however, this may also be achieved via a venous injection of the contrast medium wherein the opacification is reduced. In both cases, the datasets produced may be difficult to interpret because nidi are complex structures. In addition, the mixing of the contrast medium in different afferent vessels of the nidus make interpretation more difficult.

### SUMMARY AND DESCRIPTION

It is therefore an object of the present disclosure to enable an improved acquisition of a dynamic behavior of contrast medium flows in branched hollow organs.

The scope of the present disclosure is defined solely by the appended claims and is not affected to any degree by the statements within this summary. The present embodiments may obviate one or more of the drawbacks or limitations in the related art.

In a first aspect, a computer-implemented method for providing a results database is disclosed. In act a) of the method, an image dataset of an examination object is acquired. The image dataset maps a contrast medium flow in a branched hollow organ of the examination object spatially and temporally resolved. In act b), portions of the branched hollow organ are identified in the image dataset on the basis of the mapped contrast medium flow. In act c), sub-portions of the identified portions are classified into single-fed and multi-fed sub-portions on the basis of a mapped flow direction of the contrast medium flow and an identification of confluences of the identified portions of the branched hollow organ that are mapped in the image dataset. Therein, sub-portions of the branched hollow organ that are arranged downstream relative to a confluence are classified as multi-fed sub-portions. In act d), the resulting dataset is provided on the basis of the image dataset in the classified sub-portions of the branched hollow organ. The resulting dataset has a partial dataset for each of the classified sub-portions of the branched hollow organ. The partial datasets have a dedicated representation of the contrast medium flow in each classified sub-portion of the branched hollow organ.

The acts described above may advantageously be carried out at least partially simultaneously or consecutively.

The acquisition of the image dataset may include an acquisition and/or a readout of a computer-readable data store and/or a reception from a data storage unit, for example, a database. Additionally, the image dataset may be provided by a provision unit of a medical imaging device for recording the image dataset. The medical imaging device may include a magnetic resonance tomography (MRT) system, a computed tomography (CT) system, a medical X-ray device, (e.g., a medical C-arm X-ray device), an ultrasound device, a positron emission tomography (PET) system, or a combination thereof.

The image dataset may include a spatially and temporally resolved mapping of the examination region of the examination object, in particular, the contrast medium flow in the branched hollow organ of the examination object. Therein, the image dataset may be spatially resolved, in particular, two-dimensionally (2D) and/or three-dimensionally (3D). Advantageously, the image dataset may map a flow, in particular, a spreading movement and/or a flow movement of the X-ray opaque contrast medium arranged in the branched hollow organ. The image dataset may have a plurality of image points, in particular pixels and/or voxels, each having a time-intensity curve. The time-intensity curves may map the temporal variation of image values, in particular, intensity values and/or attenuation values of the respective image points. The branched hollow organ may include an, in particular, arterial and/or venous, vascular tree and/or at least a part of a vascular malformation, in particular, a vascular malformation. The vascular malformation may be a nidus and/or an aneurysm.

In particular, the branched hollow organ may be arranged on or in a body part and/or organ of the examination object. For example, the branched hollow organ may include a vascular tree of a head, in particular, a brain of the examination object.

The identification of the portions of the branched hollow organ, for example, arterial and/or venous vascular portions, in the image dataset may include a segmentation of image points that map each portion. The segmentation may be based upon a comparison of image values of the image points of the image dataset with a predetermined threshold value. Therein, in particular, the image points of the image dataset may be identified as image points mapping a portion of the branched hollow organ which map the contrast medium, in particular, the contrast medium flow. Each of the portions of the branched hollow organ may therein denote a spatially connected region of the branched hollow organ. The identification of the portions of the branched hollow organ may include an identification of a connected set of image points of the image dataset which map the respective portion of the branched hollow organ.

Advantageously, sub-portions of the identified portions of the branched hollow organ may be classified, in particular differentiated, into single-fed and multi-fed sub-portions. Advantageously, the identified portions of the branched hollow organ may each have at least one sub-portion, in particular, a plurality of sub-portions. For example, one of the identified portions may have a plurality of sub-portions, each adjoining a branching site, for example, a bifurcation or a vascular malformation in pairs. In particular, the respectively identified portion may be formed by one or more, in particular connected, sub-portions.

Advantageously, on the basis of the time-intensity curves of the image points of the image dataset that map the portions of the branched hollow organ, a flow direction of the contrast medium flow may be identified, for example, by way of a temporal comparison of an intensity change mapped in each of the time-intensity curves. In particular, for each of the image points that map one of the portions of the branched hollow organ, a time point of a contrast medium influx, in particular, a bolus arrival time may be determined on the basis of the respective time-intensity curve. By comparing the time points of the contrast medium influx, in particular, the bolus arrival times, a flow direction of the contrast medium flow may be identified for each of the portions of the branched hollow organ.

Furthermore, confluences of the identified portions of the branched hollow organ may be identified at branching sites. Confluences may herein denote branching sites of the hollow organ that have at least three sub-portions of the hollow organ, wherein the contrast medium flow is directed in at least two of the at least three sub-portions to the branching site, in particular, in a feeding manner. The at least one further portion of the hollow organ that includes the branching site and its contrast medium flow directed away from the branching site, in particular, in a discharging manner, may be classified as a multi-fed sub-portion. In particular, the at least one multi-fed sub-portion may be arranged downstream relative to the confluence, in particular, the branching site and the at least two feeding sub-portions. Sub-portions of the branched hollow organ that are not arranged downstream relative to a confluence may be classified as single-fed sub-portions.

The provision of the results dataset may include a storage on a computer-readable storage medium, a display of a graphic representation of the results dataset on a display unit, a transfer to a provision unit, or a combination thereof. In particular, a graphical representation of the results dataset may be displayed by the display unit.

Advantageously, the results dataset may be provided on the basis of the image dataset and the classified sub-portions of the hollow organ, in particular, the sub-portions of the hollow organ classified as single-fed or multi-fed. Therein, the results dataset may have a partial dataset for the classified sub-portions of the hollow organ, in particular, for each of the classified sub-portions of the hollow organ. The partial datasets may each have a dedicated representation, for example, a mapping and/or a model of the contrast medium flow in each classified sub-portion of the hollow organ. The model may include a centerline model, a volume network model, a vector model, a tensor model, or a combination thereof. The partial datasets may each have data points, in particular image points, with time-intensity curves which represent, in particular, map the contrast medium flow in the respective sub-portion. The partial datasets may each be spatially resolved as 2D or 3D. In addition, the partial datasets may each be temporally resolved. In particular, each of the partial datasets may represent only the contrast medium flow in the respective sub-portion of the hollow organ.

The proposed method may therefore advantageously enable an improved acquisition of a dynamic behavior of contrast medium flows in branched hollow organs. By way of the proposed method, a recording duration and/or burden, in particular, an X-ray dose burden on the examination object for mapping complex vascular malformations, in particular AVMs, may advantageously be reduced. Furthermore, a fusion of a plurality of recordings, in particular, a fusion of 3D or 4D DSAs may advantageously be omitted, which also leads to a workflow shortening. The separation into partial datasets may reduce a complexity of an analysis of complex vascular malformations, in particular, also in the case of overlapping portions of the branched hollow organ. Medical operating personnel may advantageously evaluate any desired number of portions of the branched hollow organ separately or in combination, for example, by way of selective switching on or off of a graphical representation of the relevant portion.

In a further advantageous embodiment of the proposed method, the hollow organ may include a vascular tree. Therein, in particular, arterial and/or venous vascular portions and/or at least a part of a vascular malformation, in particular, the vascular malformation may be identified as the portions of the vascular tree.

Advantageously, the branched hollow organ may include a vascular tree, in particular, a branched structure including a plurality of vascular portions which are at least partially connected to one another at branching points. The plurality of vascular portions may include arterial and/or venous vascular portions. In addition, the vascular tree may include a vascular malformation, for example, a nidus and/or an aneurysm as a portion.

The proposed method may advantageously enable an improved acquisition of a dynamic behavior of contrast medium flows in branched hollow organs.

In a further advantageous embodiment of the proposed method, the partial datasets may have a spatially and temporally resolved representation of the contrast medium flow in each classified sub-portion of the hollow organ.

The partial datasets may each have a dedicated representation, (e.g., a 2D or 3D spatially and temporally resolved representation), for example, a mapping and/or a model of the contrast medium flow in each classified sub-portion of the hollow organ. The mapping may map, for example, a contrast medium concentration in the respective classified sub-portion at different time points. The model may model, in particular, reproduce, for example, a contrast medium kinetics in the respective classified sub-portion, for example, on the basis of a physical or empirical model.

The proposed embodiment may advantageously enable an improved, in particular, dedicated acquisition of a dynamic behavior of contrast medium flows in sub-portions of a branched hollow organ.

In a further advantageous embodiment of the proposed method, the dedicated representations of the contrast medium flow in each classified sub-portion of the hollow organ may have a visual distinguishing feature.

The visual distinguishing feature may include a color coding, an intensity coding, (e.g., a gray-scale coding), an annotation, at least a partial transparency, or a combination thereof. Advantageously, the results dataset may be provided such that the dedicated representations of the contrast medium flow in the various sub-portions of the hollow organ may be distinguished on the basis of the visual distinguishing feature. For example, the plurality of sub-portions each have a different color coding and/or intensity coding and/or annotation and/or at least partial transparency. The visual distinguishing feature may also be configured to the classification of the respective sub-portion.

The proposed embodiment may enable an improved distinguishability of each dynamic behavior of the contrast medium flows in the sub-portions of the branched hollow organ.

In a further advantageous embodiment of the proposed method, the results dataset may include an at least partial overlaying and/or nesting and/or composition of the plurality of partial datasets.

Advantageously, the results dataset may include an at least partial, in particular, complete overlaying of the plurality of partial datasets. Therein, the regions of the plurality of partial datasets that each represent none of the plurality of sub-portions may advantageously be at least partially, in particular completely, transparently overlaid. Alternatively, or additionally, the results dataset may include a composition, (e.g., a joining together, a merging, a reconstruction, or a combination thereof), of the plurality of partial datasets. Therein, the overlaying and/or nesting and/or composition of the plurality of partial datasets may advantageously take place in such a way that the sub-portions are arranged relative to one another according to their respective arrangement in the image dataset, in particular their respective anatomical arrangement.

The proposed embodiment may advantageously enable an acquisition, (e.g., a simultaneous acquisition), of a dynamic behavior of contrast medium flows in the plurality of sub-portions of a branched hollow organ.

In a further advantageous embodiment of the proposed method, the image dataset may have a plurality of image points, each with a time-intensity curve. The identification of the portions of the branched hollow organ may therein include an identification of a contrast medium influx on the basis of the time-intensity curve of the respective image points.

The time-intensity curves of the image points of the image dataset may each map a temporal variation of intensity values, in particular, the image values of the image points of the image dataset. Advantageously, for each of the image points, a contrast medium influx, in particular, a time point of the contrast medium influx, for example, a bolus arrival time may be determined on the basis of the respective time-intensity curve. The contrast medium influx, in particular, the time point of the contrast medium influx, may be identified, for example, on the basis of a time point of a rise, in particular, a positive gradient of the respective time-intensity curve and/or an exceeding of a predetermined threshold value by the respective time-intensity curve. Advantageously, image points, the time-intensity curves of which have a mapping of a contrast medium influx may be identified, in particular, segmented as image points mapping a portion of the branched hollow organ. Furthermore, the portions, in particular, a respective mapping of the portions in the image dataset may be identified on the basis of the identified image points.

The proposed embodiment may enable a particularly precise identification of the portions of the branched hollow organ.

In a further advantageous embodiment of the proposed method, act c) may include a classification of at least a part of the sub-portions into feeding and discharging sub-portions on the basis of a flow direction of the contrast medium mapped in edge-region image points of the image dataset. The edge-region image points may map the branched hollow organ in an edge region of the image dataset. Therein, the classification of the sub-portions into single-fed and multi-fed sub-portions may additionally be based upon the classification of the at least a part of the sub-portions as feeding and discharging.

Advantageously, sub-portions of the branched hollow organ that are mapped by edge-region image points of the image dataset may be identified. Edge-region image points may include image points of the image dataset that are arranged within an, in particular spatial, edge region of the image dataset and each map at least one sub-portion of the branched hollow organ. The edge region of the image dataset may be predetermined, in particular defined, by way of a spatial delimitation, in particular, a delimitation contour and/or a delimitation area of a volume mapped by the image dataset and/or an area of the examination object mapped by the image dataset. Therein, the edge-region image points may include the image points of the image dataset that map the at least one sub-portion of the branched hollow organ which directly adjoin the delimitation contour and/or the delimitation area and/or are arranged within a predetermined spatial distance relative to the delimitation contour and/or the delimitation area.

Advantageously, the sub-portions of the branched hollow organ that are mapped by edge-region image points of the image dataset are identified as sub-portions of the branched hollow organ that are partially captured in the mapping. Furthermore, for each of the partially captured sub-portions of the hollow organ, a flow direction of the contrast medium may be identified, in particular, on the basis of time-intensity curves of the image points that map the respective partially captured sub-portion. If the mapped flow direction of the contrast medium of one of the partially captured sub-portions is directed away from the edge region, the respective partially captured sub-portion may be classified as a feeding sub-portion. If the mapped flow direction of the contrast medium of one of the partially captured sub-portions is directed toward the edge region, the respective partially captured sub-portion may be classified as a discharging sub-portion.

Advantageously, the classification of the identified portions into single-fed and multi-fed sub-portions may additionally be based upon the classification of the at least a part of the sub-portions as feeding and discharging. In particular, feeding sub-portions may be excluded from the classification as multi-fed sub-portions.

The proposed embodiment may advantageously enable an improved classification of the identified portions into single-fed and multi-fed sub-portions.

In a further advantageous embodiment of the proposed method, for each of the edge-region image points, a time point of the contrast medium influx may be identified on the basis of the time-intensity curve. Therein, act b) may include a comparison of the respective time points of the contrast medium influx. In addition, sub-portions having earlier time points of contrast medium influx may be classified as feeding sub-portions and sub-portions having later time points of contrast medium influx may be classified as discharging sub-portions.

Advantageously, for each of the edge-region image points that map one of the sub-portions of the branched hollow organ, a time point of a contrast medium influx, in particular, a bolus arrival time is determined on the basis of the respective time-intensity curve. By way of a comparison of the time points of the contrast medium influx, (e.g., bolus arrival times), sub-portions with relatively early and relatively late time points of contrast medium influx, (e.g., bolus arrival times), may be identified. Sub-portions of the branched hollow organ that have a relatively early time point of contrast medium influx, in particular, an early bolus arrival time may be classified as feeding sub-portions. Furthermore, sub-portions of the branched hollow organ that have a comparatively late time point of contrast medium influx, in particular, a late bolus arrival time may be classified as discharging sub-portions.

The proposed embodiment may advantageously enable an improved classification of the identified portions into single-fed and multi-fed sub-portions.

In a further advantageous embodiment of the proposed method, act b) may include an application of a connected component analysis to the image dataset. Therein, the image points of the image dataset may be identified that map the portions of the branched hollow organ. Therein, the partial datasets may be provided on the basis of the identified image points.

Advantageously, the analysis of connected components (i.e., connected component analysis) may be based upon a graph theory. Therein, the analysis may be carried out beginning from a starting image point of the image points of the image dataset. The starting image point may include an edge-region image point that maps the contrast medium flow. The edge-region image point may include an image point of the image dataset that directly adjoins the delimitation contour and/or the delimitation area and/or is arranged within a predetermined spatial distance relative to the delimitation contour and/or the delimitation area. The starting image point may be identified, for example, by way of segmentation. The segmentation may be based upon a comparison of image values of the image points of the image dataset with a predetermined threshold value. Proceeding from the starting image point, on the basis of the connected component analysis, in each case, a plurality of image points of the image dataset arranged, in particular, at least pair-wise adjacent to one another may be identified which map one of the portions of the branched hollow organ. Advantageously, the identification of the image points along a portion of the branched hollow organ may be stopped if a further edge-region image point is identified as an image point mapping the portion, in particular, the contrast medium flow.

Advantageously, by way of repeated and/or parallel, in particular, simultaneous use of the connected component analysis on the image dataset, all the portions of the branched hollow organ mapped therein, in particular, the respectively associated image points of the image dataset, may be identified.

Therein, the partial datasets may be provided on the basis of the identified image points. In particular, the partial datasets may include the image points identified in relation to each classified sub-portion.

The proposed embodiment may advantageously enable an improved identification of the portions of the branched hollow organ, in particular, also where there is an at least partial overlapping of portions of the branched hollow organ.

In a further advantageous embodiment of the proposed method, common image points of the identified image points may be identified along the mapped flow direction of the contrast medium flow into the identified portions, the identified image points mapping at least two of the identified portions. Therein, sub-portions of the identified portions which are mapped by way of common image points may be classified as multi-fed sub-portions of the branched hollow organ.

At least a part of the identified portions of the branched hollow organ may have at least one branching site, in particular, a plurality of branching sites. Advantageously, common image points of the identified image points may be identified along the mapped flow direction of the contrast medium flow into the identified portions, the identified image points mapping at least two of the identified portions. In other words, the identified image points that map a common sub-portion of at least two partially different portions of the branched hollow organ may be identified as common image points. The common sub-portion that is mapped by way of the common image points may be classified as a multi-fed sub-portion of the branched hollow organ.

The proposed embodiment may advantageously enable an improved classification of the identified portions into single-fed and multi-fed sub-portions.

In a further advantageous embodiment of the proposed method, act a) may include an acquisition of a mask dataset of the examination object that maps the examination object without the contrast medium flow in the branched hollow organ. In addition, the acquisition of the image dataset may include an acquisition of a fill dataset of the examination object that maps the examination object with the contrast medium flow in the branched hollow organ. Furthermore, the image dataset may be provided as a difference image dataset from the fill dataset and the mask dataset.

The acquisition of the mask dataset and of the fill dataset may include, in particular, an acquisition and/or readout of a computer-readable data store and/or a reception from a data storage unit, for example, a database. Additionally, the mask dataset and the image dataset may be provided by a provision unit of a medical imaging device for recording the mask dataset and the fill dataset. The medical imaging device may include a magnetic resonance tomography (MRT) system, a computed tomography (CT) system, a medical X-ray device, (e.g., a medical C-arm X-ray device), an ultrasound device, a positron emission tomography (PET) system, or a combination thereof.

The mask dataset may map the examination object, in particular, the hollow organ within a first temporal phase, in particular a mask phase. Therein, within the first temporal phase, advantageously no contrast medium is situated in the examination object, in particular, the branched hollow organ. The fill dataset may map the examination object, in particular, the branched hollow organ in a second temporal phase, in particular a fill phase. Therein, within the second temporal phase, the contrast medium is situated in the examination object, in particular the branched hollow organ. In particular, during the second temporal phase, the contrast medium flows, starting from an injection site on the examination object, through the branched hollow organ.

The mask dataset and the fill dataset may map the examination object, in particular, the branched hollow organ within the respective temporal phase spatially resolved in 2D or 3D. In addition, the mask dataset and/or the fill dataset may map the examination object, in particular, the branched hollow organ temporally resolved.

The provision of the image dataset may include a subtraction of the mask dataset and the fill dataset image point by image point. Therein, the image dataset may be provided as a difference image dataset, in particular, a subtraction image dataset from the fill dataset and the mask dataset.

The proposed embodiment may advantageously enable an improved identification of the portions of the branched hollow organ.

In a further advantageous embodiment of the proposed method, act a) may include an acquisition of a plurality of projection mappings of the examination object from different projection directions. Therein, the image dataset may be reconstructed from the plurality of projection mappings.

Advantageously, the imaging device for recording the projection mappings may have a source, in particular, an X-ray source and a detector, in particular, an X-ray detector. Therein, the source and the detector may be arranged in a defined arrangement to one another, in particular, opposite one another. Furthermore, the defined arrangement of the source and the detector may be mounted able to be moved, in particular rotated and/or translated, for example, in relation to the examination object. The source may be configured for emitting radiation, in particular, X-ray radiation for transirradiating the examination object. In particular, the source may be configured for emitting a conical beam or a fan beam for transirradiating the examination object. Therein, a central ray and/or middle ray may define a projection direction of the radiation emitted from the source. For this purpose, the detector may be configured to detect the radiation, in particular, following an interaction with the examination region of the examination object. Furthermore, the detector may be configured to provide the projection mappings dependent upon the detected radiation.

The imaging device may be configured, in particular, as an X-ray device, (e.g., as a C-arm X-ray device or an O-arm X-ray device), or as a computed tomography (CT) system.

Advantageously, the image dataset may be reconstructed from the plurality of projection mappings, for example, by a back projection (e.g., filtered back projection).

The proposed embodiment may advantageously enable a 3D acquisition of the dynamic behavior of the contrast medium flows in the branched hollow organ.

In a second aspect, a provision unit is provided that is configured for carrying out a proposed method for providing a results dataset.

The advantages of the proposed provision unit correspond to the advantages of the proposed computer-implemented method for providing a results dataset. Herein, features, advantages, or alternative embodiments may also be transferred to the other claimed subject matter and vice versa.

The provision unit may advantageously include an interface, a computing unit, a storage unit, or a combination thereof. The interface, computing unit, and storage unit may be configured to carry out the acts a) to d) of the proposed method for providing a results dataset. In particular, the interface may be configured for carrying out the acts a) and d). Furthermore, the computing unit and/or the storage unit may be configured for carrying out the acts b) and c).

In a third aspect, a medical imaging device including a provision unit is disclosed. Therein, the imaging device may be configured for acquiring the image dataset. The advantages of the proposed provision unit correspond to the advantages of the proposed method for providing a results dataset and/or the proposed provision unit. Herein, features, advantages or alternative embodiments may also be transferred to the other claimed subject matter and vice versa.

The medical imaging device may include a magnetic resonance tomography (MRT) system, a computed tomography (CT) system, a medical X-ray device, (e.g., a medical C-arm X-ray device), an ultrasound device, a positron emission tomography (PET) system, or a combination thereof.

Advantageously, the imaging device may be configured for acquiring, in particular, recording the image dataset.

In a fourth aspect, a computer program product is provided. The computer program product includes a computer program configured to be loaded directly into a memory store of a provision unit, having program portions in order to carry out all the acts of a proposed method for providing a results dataset when the program portions are executed by the provision unit.

The computer program product may include an item of software with a source code that is compiled and linked or may only be interpreted, or an executable software code that, for execution, is loaded into the provision unit. By the computer program product, the method for providing a results dataset by a provision unit may be carried out rapidly, exactly reproducibly, and robustly. The computer program product is configured such that the computer program product may carry out the method acts described herein by the provision unit.

The computer program product is stored, for example, on a non-transitory computer-readable storage medium or is deposited on a network or server from where the computer program product may be loaded into the processor of a provision unit, which may be directly connected to the provision unit, or which may be configured as part of the provision unit. Furthermore, control information of the computer program product may be stored on an electronically readable data carrier. The control information of the electronically readable data carrier may be configured such that it carries out a method when the data carrier is used in a provision unit. Examples of electronically readable data carriers are a DVD, a magnetic tape, or a USB stick, on which electronically readable control information, in particular, software, is stored. If these items of control information are read from the data carrier and stored in a provision unit, all the embodiments of the above-described methods may be carried out.

A realization through software has the advantage that conventionally used provision units may also easily be upgraded with a software update in order to operate in the manner according to the disclosure. Such a computer program product may include, where relevant, in addition to the computer program, additional constituents, such as, for example, documentation and/or additional components as well as hardware components, for example, hardware keys (dongles, etc.) in order to use the software.

### BRIEF DESCRIPTION OF THE DRAWINGS

Exemplary embodiments are illustrated in the drawings and are described in greater detail below. In the different figures, the same reference signs are used for the same features.

FIGS. 1 to 4 show schematic representations of different advantageous embodiments of a proposed method for providing a results dataset.

FIG. 5 shows a schematic representation of an advantageous embodiment of a proposed provision unit.

FIG. 6 shows a schematic representation of an exemplary identification of portions and classifications of sub-portions of the identified portions of a branched hollow organ.

FIG. 7 shows a schematic representation of an advantageous embodiment of a proposed medical imaging device as a medical C-arm X-ray device.

### DETAILED DESCRIPTION

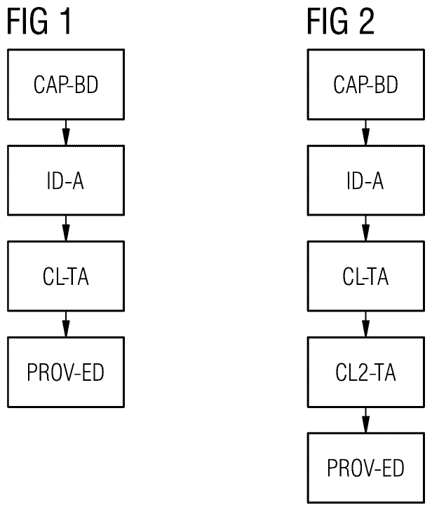

FIG. 1 shows a schematic representation of an advantageous embodiment of a proposed method for providing a results dataset PROV-ED. In act a) of the method, an image dataset of an examination object may be acquired CAP-BD. Therein, the image dataset may map a contrast medium flow in a branched hollow organ of the examination object spatially and temporally resolved.

In a further act b), portions of the branched hollow organ may be identified ID-A in the image dataset on the basis of the mapped contrast medium flow. For example, the image dataset may have a plurality of image points, each with a time-intensity curve. The identification of the portions ID-A of the branched hollow organ may therein include an identification of a contrast medium influx on the basis of the time-intensity curves of the respective image points.

In a further act c), sub-portions of the identified portions are classified CL-TA into single-fed and multi-fed sub-portions on the basis of a mapped flow direction of the contrast medium flow and an identification of confluences of the identified portions of the hollow organ that are mapped in the image dataset. Therein, sub-portions of the branched hollow organ that are arranged downstream relative to a confluence are classified CL-TA as multi-fed sub-portions.

In a further act d), the results dataset is provided PROV-ED on the basis of the image dataset and the classified sub-portions of the branched hollow organ. Therein, the results dataset may have a partial dataset for each of the classified sub-portions of the hollow organ. Furthermore, the partial datasets may have a dedicated representation of the contrast medium flow in each classified sub-portion of the branched hollow organ.

For example, the branched hollow organ may include a vascular tree. Therein, in particular, arterial and/or venous vascular portions and/or at least a part of a vascular malformation, in particular, a vascular malformation may be identified ID-A as the portions of the vascular tree. Furthermore, the partial datasets may have a spatially and temporally resolved representation of the contrast medium flow in each classified sub-portion of the hollow organ. Therein, the dedicated representations of the contrast medium flow in each classified sub-portion of the branched hollow organ may have a visual distinguishing feature. Furthermore, the results dataset may include an at least partial overlaying and/or nesting and/or composition of the plurality of partial datasets.

Advantageously, act b) may include an application of a connected component analysis to the image dataset. Therein, the image points of the image dataset may be identified that map the identified portions of the branched hollow organ. Furthermore, the partial datasets may be provided on the basis of the identified image points. Further, common image points of the identified image points may be identified along the mapped flow direction of the contrast medium flow in the identified portions, the identified image points mapping at least two of the identified portions. Therein, sub-portions of the identified portions which are mapped by way of common image points may be classified CL-TA as multi-fed sub-portions of the branched hollow organ.

FIG. 2 shows a schematic representation of a further advantageous embodiment of a proposed method for providing a results dataset PROV-ED. Therein, act c) may include a classification CL2-TA of at least a part of the sub-portions into feeding and discharging sub-portions on the basis of a flow direction of the contrast medium mapped in edge-region image points of the image dataset. Furthermore, the edge-region image points may map the branched hollow organ in an edge region of the image dataset. Furthermore, the classification CL-TA of the sub-portions into single-fed and multi-fed sub-portions may additionally be based upon the classification CL2-TA of the at least a part of the sub-portions as feeding and discharging.

Furthermore, for each of the edge-region image points, a time point of the contrast medium influx may be identified on the basis of the time-intensity curve. Therein, act b) may include a comparison of the respective time points of the contrast medium influx. Furthermore, the sub-portions having earlier time points of the contrast medium influx may be classified as feeding sub-portions and sub-portions having later time points of contrast medium influx may be classified CL2-TA as discharging sub-portions.

An exemplary method sequence is described below.

In the exemplary method, a 3D or 4D-DSA dataset including a plurality of projection mappings may be recorded by the medical imaging device, for example, a medical C-arm X-ray device, wherein during a fill phase, a contrast medium is situated within the examination object. The contrast medium may have been injected into the branched hollow organ, in particular, an aorta or vein of the examination object before the start of the fill phase. For example, the injection of the contrast medium may have been undertaken according to an injection protocol in accordance with the publication by Klostranec, Jesse M., et al., “Comparison of aortic arch and intravenous contrast injection techniques for C-arm cone beam CT: implications for cerebral perfusion imaging in the angiography suite,” Academic radiology 20.4 (2013), pp. 509-518.

In a further act, a 3D-DSA reconstruction of the projection mappings may be carried out in order to obtain a complete representation of a contrasted vasculature of a head, in particular, a brain of the examination object. Therein, the vasculature, in particular, a vascular tree of the head of the examination object may represent the branched hollow organ of the examination object.

In a further act, a 4D-DSA reconstruction may be carried out, making use of the projection mappings, for example, by a plausibility-based flow condition, wherein the image dataset is provided. The acts described above may be included by the acquisition of the image dataset CAP-BD. Furthermore, the portions of the vasculature in the image dataset may be identified ID-A on the basis of the mapped contrast medium flow. Furthermore, sub-portions of the identified portions may be classified CL-TA into single-fed and multi-fed sub-portions on the basis of a mapped flow direction of the contrast medium flow and an identification of confluences of the identified portions of the vasculature that are mapped in the image dataset. Furthermore, a time information item may be assigned to each image point or defined sub-element of the image dataset.

In a further act, a time point of a contrast medium influx, in particular, a bolus arrival time may be assigned to each image point of the image dataset, in particular, by a plausibility-based flow condition, wherein the time point of the contrast medium influx is determined for each image point.

In a further act, feeding and discharging sub-portions of the vasculature that cross a field of view (FOV), for example, in the head, in particular arteries entering and/or veins leaving the brain and/or the head of the examination object may be classified CL2-TA. Alternatively, or additionally, feeding sub-portions may be classified CL2-TA on the basis of the temporal information, for example, on the basis of a comparatively short bolus arrival time or on the basis of an identification of a flow direction, in particular, a rise in the bolus arrival time in adjacent image points. A 4D volume may be calculated for each of the feeding sub-portions. Therein, a connected component analysis may be applied to an edge-region image point of the feeding sub-portion as the starting point. Therein, a plurality of volumes, in particular, a volume for each of the feeding sub-portions, may be identified. Therein, the volumes may have a certain amount of overlap, for example, in a region of a vascular malformation, in particular, a nidus and/or efferent veins.

In a further act, the overlap of these volumes may be determined. In particular, the common image points that map more than one of the connected sub-portions may be identified.

In a further act, the sub-portions mapped in the volume may be separated into subvolumes. In particular, a partial dataset may be provided for each of the classified sub-portions of the branched vasculature. Therein, the partial datasets may map a respective subvolume. The distinguishing may therein include feeding sub-portions before a connection to other feeding sub-portions and multi-fed sub-portions.

In a further act, a graphical representation of the subvolumes may be provided individually or together, for example, color-coded. Therein, each image impression of the graphical representation of the subvolumes in a postprocessing may have been processed differently, for example, by way of a windowing in order to compensate for different mixtures of blood and contrast medium in the various feeding sub-portions. For a future, in particular, planned embolization treatment by an arterial access, for example, the mappings of the feeding sub-portions may be provided as an overlaying of a momentary mapping and/or representation of an examination region of the examination object during the procedure.

By way of the proposed method, a number of recordings, in particular, a recording duration and an X-ray dose for mapping complex AVMs may advantageously be reduced. Furthermore, a fusion of a plurality of 3D recordings may advantageously be omitted, which also leads to a workflow shortening. The separation into partial datasets may reduce the complexity of an analysis of the complex AVM structures, in particular, also with overlapping portions. Medical operating personnel may advantageously evaluate any desired number of portions of the branched hollow organ separately or in combination, for example, by way of selective switching on or off of a graphical representation of the relevant portion.

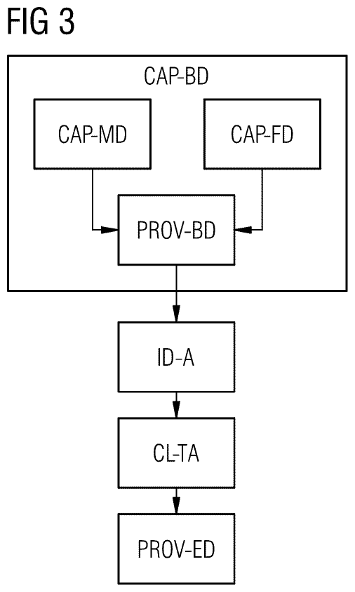

FIG. 3 shows a schematic representation of a further advantageous embodiment of a proposed method for providing a results dataset PROV-ED. Therein, act a) may include an acquisition of a mask dataset CAP-MD of the examination object which maps the examination object without the contrast medium flow in the hollow organ. Furthermore, act a) may include an acquisition of a fill dataset CAP-FD of the examination object which maps the examination object with the contrast medium flow in the hollow organ. In addition, act a) may include a provision of the image dataset PROV-BD as a difference image dataset from the fill dataset and the mask dataset.

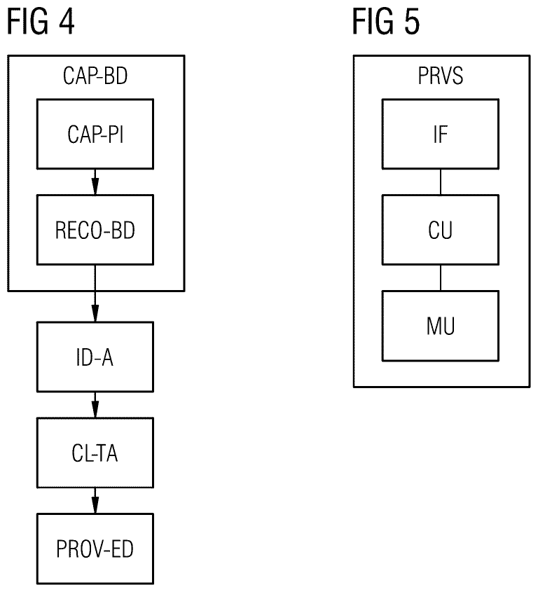

FIG. 4 shows a schematic representation of a further advantageous embodiment of a proposed method for providing a results dataset PROV-ED. Therein, act a) may include an acquisition of a plurality of projection mappings CAP-PI of the examination object from different projection directions. Furthermore, the image dataset may be reconstructed from the plurality of projection mappings RECO-BD.

FIG. 5 shows a schematic representation of a proposed provision unit PRVS. Therein, the provision unit PRVS may include a computing unit CU, a storage unit MU and/or an interface unit IF. The provision unit PRVS may be configured for carrying out a proposed method for providing a results dataset in which the interface IF, the computing unit CU and/or the storage unit MU are configured to carry out the corresponding method acts. The interface IF, the computing unit CU and the storage unit MU may be configured to carry out the acts a) to d) of the proposed method for providing a results dataset. In particular, the interface IF may be configured for carrying out the acts a) and d). Furthermore, the computing unit CU and/or the storage unit MU may be configured for carrying out the acts b) and c).

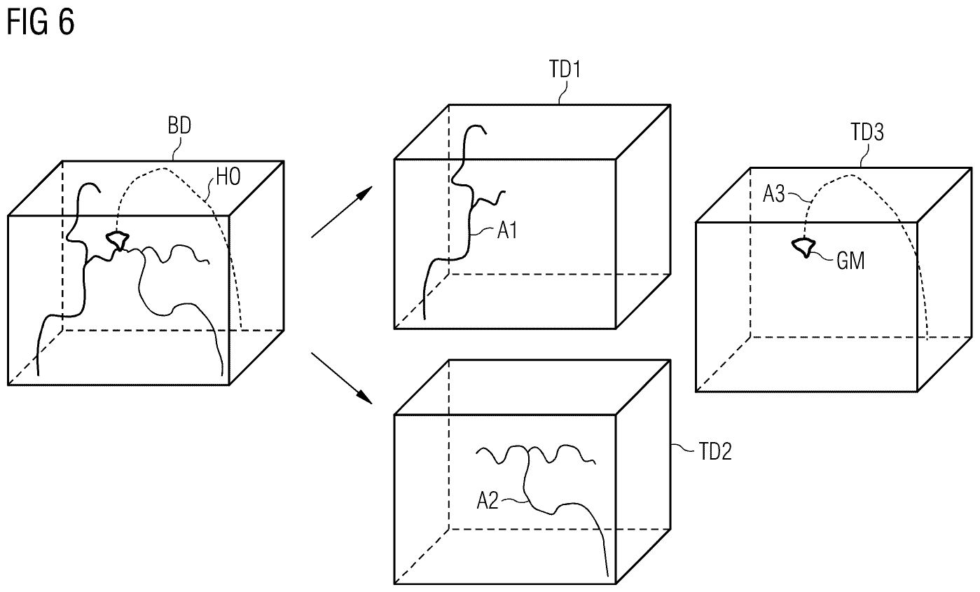

FIG. 6 shows a schematic representation of an exemplary identification of portions and classifications of sub-portions of the identified portions of a branched hollow organ HO. Therein, BD illustrates an image dataset having a 3D mapping of the branched hollow organ HO. Furthermore, three partial datasets TD1, TD2, TD3 are illustrated in FIG. 6. The first partial dataset TD1 may have a mapping of a first single-fed sub-portion A1 of an identified portion of the branched hollow organ HO, for example, a left internal carotid artery. Furthermore, the second partial dataset TD2 may have a mapping of a second single-fed sub-portion A2 of an identified portion of the branched hollow organ HO, for example, a right internal carotid artery. In addition, the third partial dataset TD3 may have a mapping of a vascular malformation GM, for example, a nidus and a multi-fed sub-portion A3 of an identified portion of the branched hollow organ HO, for example, a common vessel portion.

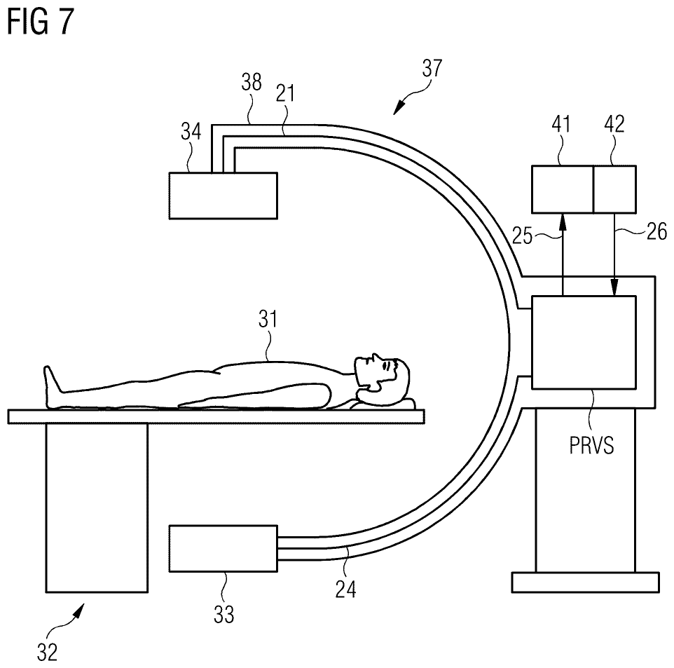

FIG. 7 shows, by way of example, for a medical imaging device, a schematic representation of a medical C-arm X-ray device 37 including a provision unit PRVS. The medical C-arm X-ray device 37 may advantageously have a detector 34, in particular, an X-ray detector and a source 33, in particular, an X-ray source, which are arranged in a defined arrangement on a C-arm 38. The C-arm 38 of the C-arm X-ray device 37 may be mounted to be movable about one or more axes. For acquiring, in particular, recording the image dataset CAP-BD of the examination object 31 arranged on a patient positioning apparatus 32, the provision unit PRVS may transmit a signal 24 to the X-ray source 33. Thereupon, the X-ray source 33 may emit an X-ray beam. When the X-ray beam impinges upon a surface of the detector 34 following an interaction with the examination object 31, the detector 34 may emit a signal 21 to the provision unit PRVS. The provision unit PRVS may acquire projection mappings of the examination object CAP-PI on the basis of the signal 21 and may reconstruct the image dataset RECO-BD. The acquisition of the image data CAP-BD may include the acquisition of the projection mappings CAP-PI and the reconstruction of the image dataset RECO-BD.

The C-arm X-ray device 37 may further have an input unit 42, for example, a keyboard, and a display unit 41, for example, a monitor and/or display and/or a projector. The input unit 42 may be integrated into the display unit 41, for example, in the case of a capacitive and/or resistive input display. The input unit 42 may advantageously be configured for acquiring a user input. For this purpose, the input unit 42 may transmit, for example, a signal 26 to the provision unit PRVS. The provision unit PRVS, in particular, the C-arm X-ray device 37 may be configured to be controlled, dependent upon the user input, in particular, the signal 26, in particular, for carrying out a method for providing a results dataset PROV-ED.

The display unit 41 may advantageously be configured to display a graphical representation of the results dataset. For this purpose, the provision unit PRVS may transmit a signal 25 to the display unit 41.

By the proposed method, vascular, in particular, neurovascular regions in a 3D or 4D reconstruction of a single rotation recording may be separated by the temporal information. Therein, before the start of the rotation recording, advantageously a contrast medium may have been injected into the aorta or a vein.

The schematic representations contained in the drawings described do not reveal any scale or size relationships.

In summary, the methods described above in detail and the apparatuses disclosed are merely exemplary embodiments that may be modified by a person skilled in the art in a wide variety of ways without departing from the scope of the disclosure. Furthermore, the use of the indefinite article “a” or “an” does not preclude the possibility that the relevant features may also be present plurally. Similarly, the expressions “unit” and “element” do not preclude the components in question including a plurality of cooperating subcomponents which may also be spatially distributed.

The expression “based upon/on the basis of” may be understood in the context of the present application, in the sense, in particular, of the expression “making use of.” In particular, a formulation according to which a first feature is generated, established, or determined based upon a second feature does not preclude the first feature being able to be generated, established, or determined based upon a third feature.

It is to be understood that the elements and features recited in the appended claims may be combined in different ways to produce new claims that likewise fall within the scope of the present disclosure. Thus, whereas the dependent claims appended below depend on only a single independent or dependent claim, it is to be understood that these dependent claims may, alternatively, be made to depend in the alternative from any preceding or following claim, whether independent or dependent, and that such new combinations are to be understood as forming a part of the present specification.

While the present disclosure has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description

1. A computer-implemented method for providing a results dataset, the computer-implemented method comprising:
acquiring an image dataset of an examination object, wherein the image dataset maps a contrast medium flow in a branched hollow organ of the examination object spatially and temporally resolved;
identifying portions of the branched hollow organ in the image dataset based on the mapped contrast medium flow;
classifying sub-portions of the identified portions into single-fed sub-portions and multi-fed sub-portions based on a mapped flow direction of the contrast medium flow and an identification of confluences of the identified portions of the branched hollow organ mapped in the image dataset, wherein each sub-portion of the branched hollow organ arranged downstream relative to a confluence is classified as a multi-fed sub-portion; and
providing the results dataset based on the image dataset and the classified sub-portions of the branched hollow organ,
wherein the results dataset has a partial dataset for each classified sub-portion of the branched hollow organ to provide partial datasets, and
wherein the partial datasets are configured to have a dedicated representation of the contrast medium flow in each classified sub-portion of the branched hollow organ.

2. The computer-implemented method of claim 1, wherein the branched hollow organ comprises a vascular tree, and
wherein arterial vascular portions, venous vascular portions, at least a part of a vascular malformation, or combinations thereof are identified as the portions of the vascular tree.

3. The computer-implemented method of claim 1, wherein the partial datasets have a spatially and temporally resolved representation of the contrast medium flow in each classified sub-portion of the branched hollow organ.

4. The computer-implemented method of claim 1, wherein the dedicated representations of the contrast medium flow in each classified sub-portion of the branched hollow organ have a visual distinguishing feature.

5. The computer-implemented method of claim 4, wherein the results dataset comprises an at least partial overlaying, nesting, composition, or combination thereof of the partial datasets.

6. The computer-implemented method of claim 1, wherein the image dataset has a plurality of image points,
wherein each image point of the plurality of image points has a time-intensity curve, and
wherein the identification of the portions of the branched hollow organ comprises an identification of a contrast medium influx based on the time-intensity curves of respective image points of the plurality of image points.

7. The computer-implemented method of claim 6, wherein, for each edge-region image point of the plurality of image points, a time point of the contrast medium influx is identified based on the respective time-intensity curve,
wherein the identifying of the portions of the branched hollow organ comprises a comparison of the respective time points of the contrast medium influx, and
wherein sub-portions having earlier time points of the contrast medium influx are classified as feeding sub-portions and sub-portions having later time points of the contrast medium influx are classified as discharging sub-portions.

8. The computer-implemented method of claim 1, wherein the classifying of the sub-portions comprises a classification of at least a part of the sub-portions into feeding sub-portions or discharging sub-portions based on a flow direction of the contrast medium mapped in edge-region image points of the image dataset,
wherein the edge-region image points map a hollow organ in an edge region of the image dataset, and
wherein the classifying of the sub-portions into the single-fed sub-portions and the multi-fed sub-portions is additionally based upon the classification of the at least part of the sub-portions into the feeding sub-portions or the discharging sub-portions.

9. The computer-implemented method of claim 8, wherein, for each edge-region image point of the plurality of image points, a time point of a contrast medium influx is identified based on the respective time-intensity curve,
wherein the identifying of the portions of the branched hollow organ comprises a comparison of the respective time points of the contrast medium influx, and
wherein sub-portions having earlier time points of the contrast medium influx are classified as the feeding sub-portions and sub-portions having later time points of the contrast medium influx are classified as the discharging sub-portions.

10. The computer-implemented method of claim 1, wherein the identifying of the portions of the branched hollow organ comprises an application of a connected component analysis to the image dataset,
wherein image points of a plurality of image points of the image dataset are identified that map the identified portions of the branched hollow organ, and
wherein the partial datasets are provided based on the identified image points.

11. The computer-implemented method of claim 10, wherein, along the mapped flow direction of the contrast medium flow into the identified portions, common image points of the identified image points are identified,
wherein each common image point maps at least two of the identified portions, and
wherein sub-portions of the identified portions mapped by way of the common image points are classified as multi-fed sub-portions of the branched hollow organ.

12. The computer-implemented method of claim 1, wherein the acquiring of the image dataset comprises:
acquiring a mask dataset of the examination object that maps the examination object without the contrast medium flow in the branched hollow organ;
acquiring a fill dataset of the examination object that maps the examination object with the contrast medium flow in the branched hollow organ; and
providing the image dataset as a difference image dataset from the fill dataset and the mask dataset.

13. The computer-implemented method of claim 1, wherein the acquiring of the image dataset comprises acquiring a plurality of projection mappings of the examination object from different projection directions, and
wherein the image dataset is reconstructed from the plurality of projection mappings.

14. A medical imaging device comprising:
a provision unit configured to:
receive or acquire an image dataset of an examination object, wherein the image dataset maps a contrast medium flow in a branched hollow organ of the examination object spatially and temporally resolved;
identify portions of the branched hollow organ in the image dataset based on the mapped contrast medium flow;
classify sub-portions of the identified portions into single-fed and multi-fed sub-portions based on a mapped flow direction of the contrast medium flow and an identification of confluences of the identified portions of the branched hollow organ mapped in the image dataset, wherein each sub-portion of the branched hollow organ arranged downstream relative to a confluence is classified as a multi-fed sub-portion; and
provide a results dataset based on the image dataset and the classified sub-portions of the branched hollow organ,
wherein the results dataset has a partial dataset for each classified sub-portion of the branched hollow organ to provide partial datasets, and
wherein the partial datasets are configured to have a dedicated representation of the contrast medium flow in each classified sub-portion of the branched hollow organ.

15. The medical imaging device of claim 14, wherein the medical imaging device is configured to acquire the image dataset of the examination object.

16. A non-transitory computer program product having a computer program configured to be loaded directly into a memory store of a provision unit, wherein the computer program, when executed by a determining system of a medical imaging device, is configured to cause the medical imaging device to:
acquire an image dataset of an examination object, wherein the image dataset maps a contrast medium flow in a branched hollow organ of the examination object spatially and temporally resolved;
identify portions of the branched hollow organ in the image dataset based on the mapped contrast medium flow;
classify sub-portions of the identified portions into single-fed and multi-fed sub-portions based on a mapped flow direction of the contrast medium flow and an identification of confluences of the identified portions of the branched hollow organ mapped in the image dataset, wherein each sub-portion of the branched hollow organ arranged downstream relative to a confluence is classified as a multi-fed sub-portion; and
provide a results dataset based on the image dataset and the classified sub-portions of the branched hollow organ,
wherein the results dataset has a partial dataset for each classified sub-portion of the branched hollow organ to provide partial datasets, and
wherein the partial datasets are configured to have a dedicated representation of the contrast medium flow in each classified sub-portion of the branched hollow organ.
